(19) United States
(12) Patent Application Publication
(10) Pub. No.: US 2005/0205100 A1
(43) Pub. Date: Sep. 22, 2005

(54) Thumb protector

(76) Inventor: Dean Jensen, Mission Viejo, CA (US)

Correspondence Address: MATTHEW A . NEWBOLES;STETINA BRUNDA GARRED & BRUCKER, SUITE 250, 75 ENTERPRISE, ALISO VIEJO, CA, 92656, US

(21) Appl. No.: 10/805,756

(22) Filed: Mar. 22, 2004

(51) Int. Cl. A41D 1/00; A61F 5/37

(52) U.S. Cl. 128/880

(57) ABSTRACT

A thumb protector comprising an upper layer attached to a lower layer at first distal ends with an attachment mechanism to connect second distal ends of the upper and lower layers is provided. The second distal ends of the layers form an aperture and have a circumference smaller than a thumb circumference to retain the thumb protector on the thumb. The lower layer is soft and elastic to disperse pressure applied to an external surface of the lower layer to thereby reduce pressure applied to the thumb to prevent soreness and callous formation while still imparting a natural sense of feel.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

The present invention relates generally to a thumb protector placed over an area of skin which is prone to soreness, developing callous formation, or experience slipping, and more particularly to a human thumb protector being fabricated with an elastic material to disperse compressive forces when playing a video game controller.

Callous is an additional layer of skin that develops on an area of the skin that is subject to repetitive frictional forces and pressures. Frequently, people develop callous when they are engaged in activity for a long duration of time. One particular area of activity where people are prone to develop callous is related to video games. Today, video games are increasing in popularity and with such popularity, video game players are playing the games for longer periods of time. Exemplary of such well-known game systems include XBOX, Playstation and Game Cube, all of which offer a wide variety of game choices that can provide countless hours of entertainment. Along these lines, the controllers for these games require that the player control the game action with a controller gripped and manipulated by their hands and fingers. As such, the hand and its fingers are subjected to long hours of repetitive frictional forces and pressures due to the manipulation of the game controller and, as such, are prone to develop callous on the hand and fingers. Generally, the most common places where such callous develop upon such individuals is the thumb.

To deal with the callous formation, game players may remove the callous after it has formed. In this regard, the health care industry markets many different types of callous removers. One common callous remover is a pumice stone which is gently rubbed against the callous and physically removes callous from the surface of the skin. This process is time consuming and generally not accepted by younger individuals most likely to play video games.

The only viable alternative to avoid a callous formation is to abstain from playing video games. As is well known, however, to refrain from such activity defeats the utility of the video game and further detracts from a player's ability to improve his or her performance. As such, there is a tremendous need in the art for a device and method of preventing callous formation on fingers and may be used during callous causing activity. The present invention attempts to eliminate callous formation by dispersing excessive pressures that may form on the fingertips, and especially the thumb, and has a unique method of retaining a protector on a finger or thumb of a user/wearer.

In addition to such objective, the present invention is further operative to address problems associated with prolonged use of game controllers and the like. Specifically, the present invention is operative to eliminate soreness in the thumb, as well as possibly other digits, that can arise from prolonged handling of video game controllers. In this regard, it is well-known that separate and apart from callous formation, the excessive application of a compressive force by the thumb and other digits, plus repetitious movement by the tip of the thumb can cause substantial soreness. Such soreness can be aggravated further to the extent a game controller is utilized over a prolonged period of time. As is well-known, prolonged use of game controllers can cause rubberized coverings over plastic parts to wear down and make the controller more difficult to operate, thus requiring a greater expenditure of force. In some cases, the loss of rubberized covering can cause plastic parts to cut into the thumb by virtue of the sharpened surfaces that become exposed as the controller parts are worn down.

Along these lines, a yet further complication that can arise through prolonged playing of game controllers that is alleviated by the present invention is the difficulty in operating a game controller when a user's hands become sweaty or if the controller surfaces actuated by the thumb become slippery. As is well-known amongst game users, often times playing video games creates tremendous amounts of stress, especially when playing at higher levels of a particularly challenging video game, particularly those that have consequences to the extent the games objectives are not achieved. In this regard, any inadvertent actuation of a game controller can cause a player to lose a game, which can and frequently does lead to intense frustration. Advantageously, however, the present invention is adapted to address such issues.

### BRIEF SUMMARY OF THE INVENTION

The present invention specifically addresses and alleviates the above-identified deficiencies in the art. In this regard, the present invention is directed to a device for preventing callous formation on the fingers and thumb of a user/wearer to thus enable such individual to engage in an activity, and in particular the manipulation of a game controller for video games, to thus protect the individual's digits during prolonged periods of play. In accordance with the present invention, there is provided a protector comprising a lower layer, upper layer and an attachment mechanism. The lower layer is positioned over the area of the thumb skin that is prone to develop callous or cause soreness. The lower layer prevents the formation of callous and provides a cushion that resists soreness over that area of the skin by dispersing any concentrated pressure applied to that area and by preventing frictional forces from rubbing against that area. In particular, the lower layer may be fabricated from a soft elastic material that is sufficiently thick. The soft elastic characteristic of the lower layer material allows the lower layer to adapt itself into the shape of the user's thumb when the protector is worn to eliminate concentrated pressure on the thumb while at the same time providing a natural sense of feel to the user. And, the thickness of the material is operative to disperse the concentrated pressure applied to the lower layer over a greater surface area on the thumb itself. Hence, the excessive pressures that the thumb may be subjected to without the appendage protector may be reduced to a great extent so as to prevent the formation of callous on the thumb.

The lower layer may define a contact area on its external surface which is a corresponding area to the thumb that is prone to develop callous. The lower layer will preferably include an external surface that, by virtue of being sufficiently padded, substantially reduces the soreness that can be experienced by thumb during prolonged periods of play. In one embodiment, the lower layer may be oversized to provide an extended reach. Moreover, the lower layer will preferably be provided with a contact area that resists slippage when engaged with the actuatable parts of a game controller to thus enable a player to correctly manipulate the device to the extent the user's hands, and particularly the thumb and/or digits become sweaty. To that end, it is contemplated that the contact area will have a texturized surface that resists slipping. The contact area of the lower layer may optionally have a tacky material or rubber or leather patch placed thereon to provide ideal friction. The tacky material may have the same coefficient of friction as that of a human skin. Or, in the alternative, the tacky material may have a coefficient of friction greater than that of the human skin to increase the gripping ability of the user. Preferably, the tacky material may be removed from the contact area of the lower layer and replaced with a fresh tacky material. This may be accomplished by either placing a washable tacky material on the contact area or by placing an adhesive layer to the contact area that may later be removed after use.

The lower layer may be connected to an upper layer at first distal ends of the lower and upper layer. The first distal end is located at the user's fingertips when the protector is worn. The upper layer is operative to form a cavity in which the thumb may be received so as to retain the lower layer at the proper position in relation to a contact area of the thumb. To form the cavity, edges of the lower and upper layers may be sown together, or may otherwise be held together through a variety of conventional means, such as by laces, zippers, snap fasteners, hook and loop fasteners, or any other devices and techniques well-known in the art. In one embodiment, the lower and upper layers are not sown together but only the first distal ends of the lower and upper layer are connected as well as second distal ends of the lower and upper layer. The latter embodiment allows for increased air flow over the contact area of the thumb and the upper side of the thumb to thereby aerate the thumb of any moisture that may be excreted from the thumb between the upper and lower layers.

As stated above, the lower layer is connected to the upper layer at the second distal ends of the layers. The connection is operative to form an aperture or opening to receive the thumb. Preferably, the opening is smaller than a diameter of the thumb to retain the protector on the thumb so that the protector may be continuously worn during the activity which would otherwise cause the callous. The opening or aperture may be smaller than the circumference of the thumb due to the elastic nature of the lower and upper layer. Simply put, the opening will stretch to the size of the user's thumb when worn.

Moreover, the opening is preferably positioned beyond the first joint of the thumb. In this way, the protector may not slip or dislodge from the thumb during the callous causing activity. The opening may be smaller than the circumference of the thumb and positioned beyond the first joint of the thumb because of the unique attachment mechanism of the present invention. The attachment mechanism connects the upper and lower layers at the corners of the second distal ends. In particular, the lower layer may have a first strap portion and a second strap portion. The first and second strap portions extend out from a longitudinal axis of the lower layer and its length is sufficient to wrap the strap portions starting from the underside of the thumb to the upper side of the thumb and onto each other.

The attachment mechanism may be strips of hooks and loops connected to the first and second straps and the upper layer. Accordingly, the straps may be adjustably wrapped around the thumb with the hooks and loops. In the alternative, the attachment mechanism may be snaps which may be connected to the first and second strap portions and the upper layer. In this embodiment, the opening circumference is not adjustable compared to the prior hooks and loops attachment mechanism but is fixed. Nonetheless, the elastic characteristic of the upper and lower layers allow the thumb protector to fit a range of large and small thumbs. Both types of attachment mechanisms are merely illustrative of the various types of attachment mechanism that may accomplish the task of connecting the upper and lower layers, and are not the exclusive methods to accomplish the same. For example, the attachment mechanism may be a button with corresponding button hole or buckle.

In further refinements of the invention, the thumb protector may be fabricated to bear a unique logo or design, or may otherwise be fabricated to be aesthetically pleasing. It is also contemplated that the thumb protector may be utilized in a variety of applications outside of video gaming, and may have utility as a general thumb protector for use in a variety of applications that involve prolonged use of the thumb. Along these lines, it is contemplated that the thumb protector may further be adapted for use by each individual digit of a user and likewise utilized for a variety of applications where it is necessary to protect the distal most ends of the digits.

### BRIEF DESCRIPTION OF THE DRAWINGS

An illustrative and presently preferred embodiment of the invention is shown in the accompanying drawings in which:

FIG. 1 is a perspective view of a person holding a video game controller with one thumb wearing a thumb protector with free edges and with the other thumb exposed to greater pressure and frictional forces compared to the thumb wearing the thumb protector;

FIG. 2 is a side view of the thumb protector of FIG. 1 with hooks and loops;

FIG. 3 is a front view of FIG. 2 showing that the hooks and loops may be adjustably engaged;

FIG. 4 is a side view of the thumb protector of FIG. 1 with hooks and loops in an engaged position around the thumb;

FIG. 5 is a front view of FIG. 4;

FIG. 6 is a top view of the thumb protector of FIG. 1 with disengaged hooks and loops;

FIG. 7 is a bottom view of the thumb protector of FIG. 6.

FIG. 8 is a perspective view of a person holding a video game controller with one thumb wearing a thumb protector with stitched edges and with the other thumb exposed to greater pressure and frictional forces compared to the thumb wearing the thumb protector;

FIG. 9 is a side view of the thumb protector FIG. 8 with snaps;

FIG. 10 is a front view of FIG. 9 indicating that a first snap receptacle is offset to a corresponding first snap protrusion;

FIG. 11 is a side view of the thumb protector of FIG. 8 with first and second snap receptacles and protrusions in an engaged position around the thumb;

FIG. 12 is a front view of FIG. 11;

FIG. 13 is a top view of the thumb protector of FIG. 8 with disengaged snaps; and

FIG. 14 is a bottom view of the thumb protector of FIG. 13.

### DETAILED DESCRIPTION OF THE INVENTION

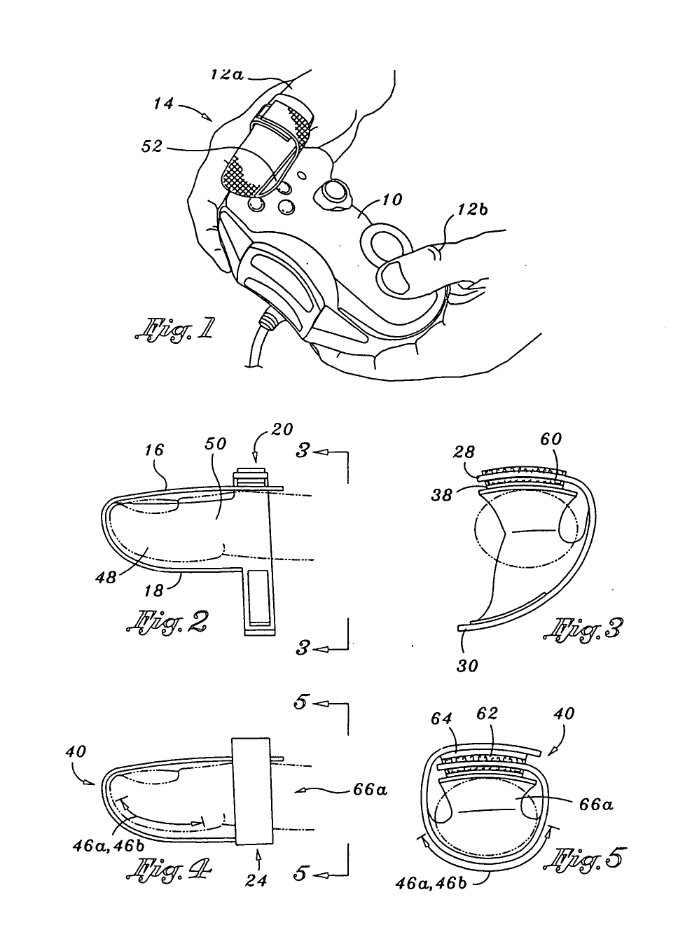

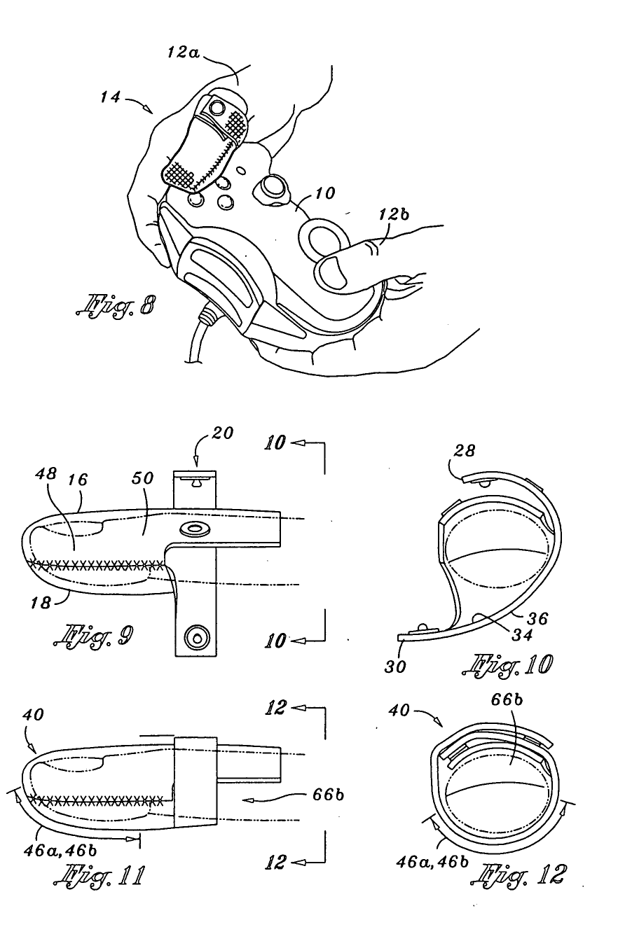

Referring now to the drawings wherein the showings are for the purposes of illustrating the preferred embodiments of the present invention only, and not for purposes of limiting the same, FIGS. 1 and 8 illustrate a perspective view of a person holding a video game controller 10 wherein only one of the thumbs 12a of the person is wearing a thumb protector 14. The thumb protector 14 which is the subject matter of the present invention may be referred to as an appendage protector 14 within this detailed description. The present invention i.e., the thumb protector 14, is directed to a device which protects skin of a human thumb from developing callous as well as prevents/resists soreness that can arise from prolonged manipulation of a video game controller. The present invention even though directed to a thumb 12 may be adapted to fit other human appendages, such as other fingers.

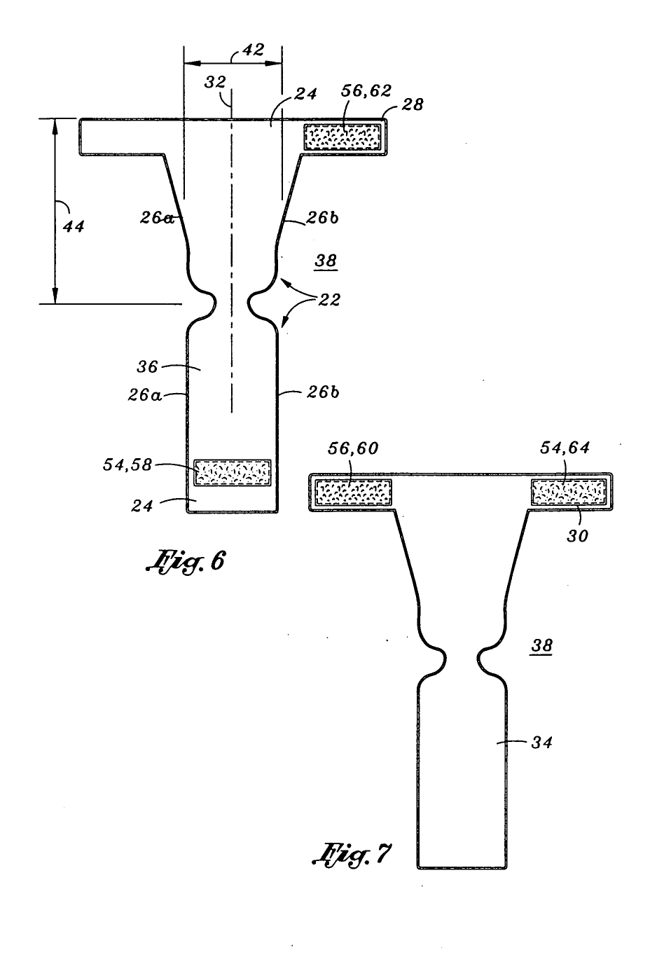

As shown in FIGS. 2 and 9, the appendage protector 14 includes an upper layer 16, lower layer 18, and an attachment mechanism 20. And, the upper and lower layers 16, 18 each define first and second distal ends 22, 24 and opposed edges 26a, 26b (see FIGS. 6 and 13). As shown in FIGS. 3 and 10, at the second distal ends 24 of the layers 16, 18, the layers 16, 18 may define first and second strap portions 28, 30. The first and second strap portions 28, 30 may extend out from a longitudinal axis 32 (see FIGS. 6 and 14) of the lower layer 18, and preferably extends out perpendicularly to the longitudinal axis 32 of the lower layer 18. The layers 16, 18 additionally define internal and external surfaces 34, 36 (see FIGS. 6, 7, and 10). The lower layer 18 and upper layer 16 define an open position 38 (see FIGS. 6 and 7) and a closed position 40 (see FIGS. 4, 5 and 11, 12). The layers 16, 18 are in the closed position 40 when the internal surfaces 34 are facing each other.

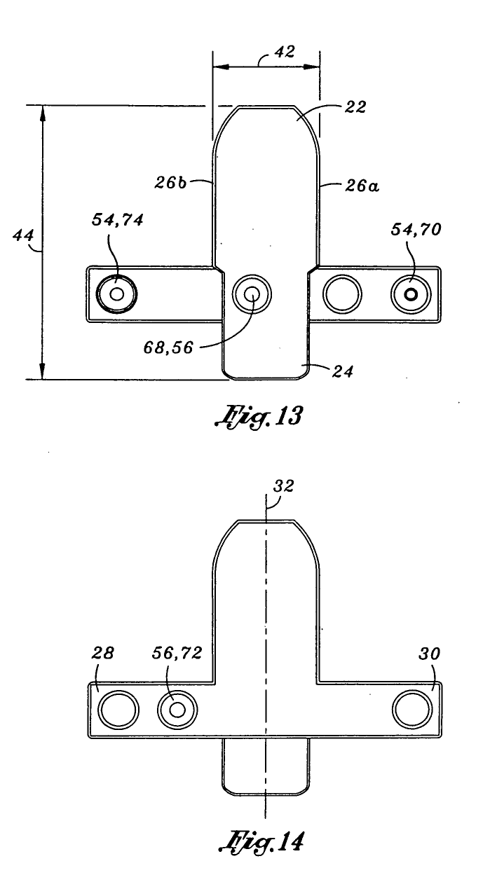

The first distal ends 22 of the upper and lower layers 16, 18 may be connected to each other. In one embodiment, the first distal ends 22 may be connected to each other by forming the upper and lower layers 16, 18 from a unitary material, as shown in FIGS. 6 and 7. In the alternative, the first distal ends 22 may be connected to each other by forming the upper and lower layers 16, 18 in two pieces and then subsequently joining the pieces to each other by sowing, stitching or other joining methods, as shown in FIGS. 9 and 11. The attachment methods described to join the first distal ends 22 are merely illustrative and not exclusive of the method of attaching the first distal ends 22, rather, any methods suitable to attach the first distal ends 22 may be employed so long as the attachment method is compatible with the material(s) of the layers 16, 18.

The first distal ends 22 may have a completely curved configuration such that the thumb 12 may fit snug within the protector 14 when the layers 16, 18 are folded upon each other. Alternatively, the first distal ends 22 may be curved on both sides of the first distal ends 22 with a flat apex (see FIGS. 13 and 14). The flat apex may be formed when the upper and lower layers are folded on each other (see FIGS. 6 and 7).

The width and length 42, 44 (see FIGS. 6 and 13) of the lower layer 18 may cover a contact area 46a (see FIGS. 4,5 and 11, 12) of the thumb 12 which is the area of the thumb 12 that is subject to the greatest degree of stress, soreness and/or callous formation. Preferably, the lower layer length 44 is greater than the first distal phalanx bone 48 of the thumb or at least extends beyond the first joint 50 of the thumb 12 when the protector 14 is placed on the thumb 12 (see FIGS. 2 and 9).

As stated above, the lower layer 18 may cover the contact area 46a of the thumb 12, and in this regard, the lower layer 18 additionally defines a contact area 46b of the lower layer which correlates to the thumb contact area 46a. The lower layer contact area 46b may be fabricated from a resilient and elastic material which may be accomplished by fabricating the complete lower layer 18 from material with the desired characteristics. Alternatively, the contact area 46b may be fabricated from a resilient and elastic material by sowing an additionally layer with the desired characteristics to the lower layer 18 at the respective area. Along these lines, it is contemplated that the contact area 46b may be fabricated to be enlarged or provide an extended reach to thus enable certain users, such as younger users, to have an extended reach to manipulate the various controls of a game controller. The lower layer contact area 46b may also have placed thereon a tacky material to provide additional gripping beyond the amount of gripping that the lower layer material may be capable of independently providing. The contact area 46b of the lower layer 18 may be cleanable in that the tacky material may be repetitively removed and fresh tacky material placed thereon. In all embodiments, however, it is preferred that the thumb protector be designed to impart a natural feel to the user to thus enable the user at all times to maintain control and have a natural feel while manipulating the controller.

The upper layer 16 and the lower layer 18 may be fabricated from identical material or different material. Preferably, the layers 16, 18 are fabricated from elastic or stretchable and/or resilient material. The layers 16, 18 are preferably fabricated from material that is comfortable on the wearer and will thus enable the protector to be worn comfortably for prolonged periods of time.

As stated above, the upper and lower layers 16, 18 define the edges 26 which extend from the first distal ends 22 to the second distal ends 24 thereof. The edges 26a, 26b of the upper and lower layers 16, 18 may be sown to each other or connectable to one another by laces, zippers, hook and loop fasteners and the like to accomplish a more snug fit between the protector 14 and thumb 12 (see FIG. 8) compared to a protector 14 wherein the edges 26 are not sown to each other (see FIG. 1). If the edges 26 are not sown together then the edges form an aperture 52 (see FIG. 1) on opposed sides of the thumb 12 to provide an escape for any moisture that may be excreted from the thumb 12.

The protector 14 may also comprise a set of snaps (see FIG. 9), or hooks and loops (see FIG. 3; commonly sold under the trademark “Velcro”). The snaps and hooks and loops will be referred to in this detailed description as the attachment mechanism 20 which includes a locking member 54 and a receptacle 56. In this regard, a receiving snap may be equivalent to the receptacle 56 and a protrusion snap may be equivalent to the locking member 54 (see FIGS. 13 and 14). Similarly, the hooks may be equivalent to the receptacle and the loops may be the locking members (see FIGS. 6 and 7). This detailed description of the present invention will discuss the attachment mechanism 20 in relation to snaps and hooks and loops but the present invention is not limited to these types of attachment mechanism 20. For example, the attachment mechanism 20 may be hooks and loops, buttons and button holes, buckle, snaps, or other attachment mechanisms 20.

In an embodiment of the present invention, the attachment mechanism 20 may be hooks and loops. In this regard, a first strip of loops 58 may be attached to the external surface 36 of the upper layer 16 at the second distal end 24 (see FIG. 6). The first strip of loops 58 may be attached to the upper layer 16 at a position greater than a first joint 50 of the thumb 12. And, a first strip of hooks 60 (see FIG. 7) and second strip of hooks 62 (see FIG. 6) may be may be attached to the first strap portion 28 on the internal and external surfaces 34, 36, respectively. The first strap portion 28 and the first strip of hooks 60 are located such that the first strips of hooks 60 may fold over to engage the first strip of loops 58 (see FIG. 3). Additionally, a second strip of loops 64 may be attached to the second strap portion 30 (see FIG. 7). The second strap portion 30 may be folded over such that the second strip of loops 64 may engage the second strip of hooks 62 (see FIG. 5).

The first and second strips of hooks and loops 58, 60, 62, 64, when engaged, connect the second distal ends 24 of the layers 16, 18 (see FIG. 4). The second distal ends 24 now form an opening 66a which is sized and is configurable to fit over the thumb 12. The hooks and loops allow the opening 66a to be adjustable so that a single protector 14 may fit over large and small thumbs 12. Additionally, the elastic characteristic of the layers 16, 18 allows the protector 14 to fit over large and small thumbs 12. The opening 66a defines a circumference and is preferably smaller compared to an outside diameter of the first joint of the thumb 12. In this way, a retainment force is created which circumscribes the thumb 12. The retainment force may be created because the material is elastic and will stretch to fit (i.e., configurable) around the thumb 12. The frictional forces between the thumb skin and the layer material provide frictional forces so that the protector 14 is retained on the thumb 12. Additionally, the strips of hooks and loops are positioned on the layers 16, 18 such that it will be positioned beyond the first joint 50 of the thumb 12 when engaged and the protector 14 is placed on the thumb 12. The opening 66a must enlarge beyond the size of the first joint 50 to remove the protector 14 after the loops and hooks are engaged which further provides additional retainment forces to retain the protector 14 on the thumb 12.

In another embodiment of the present invention, the attachment mechanism 20 may be snaps. On the second distal end 24 of the upper layer 16, a first receiving snap 68 (see FIG. 13) may be attached thereto. And, on the internal surface 34 of the first strap portion 28, a first protrusion snap 70 may be attached thereto and positioned such that the first receiving snap 68 and the first protrusion snap 70 may be engaged (see FIG. 12). On the external surface 36 of the first strap portion 28, a second receiving snap 72 may be attached thereto. And, on the internal surface 34 of the second strap portion 30, a second protrusion snap 74 (see FIG. 13) may be attached thereto such that the second protrusion and receiving snaps 74, 72 may be engaged after the first receiving and protrusion snaps 68, 70 are engaged (see FIG. 12).

The first receiving snap 68 is positioned and attached to the first strap portion 28 such that the first strap portion 28 may have to stretch slightly prior to engagement with the first protrusion snap 70. The stretch of the first strap portion is better viewed by comparing FIG. 10 to FIG. 12. In FIG. 10, the first protrusion snap 70 is not capable of engaging the first receiving snap 68 when the first strap portion 28 is in the unstretched condition. In contrast, in FIG. 12, the first protrusion snap 70 is engaged with the first receiving snap 68 when the first strap portion 28 is in the stretched condition. Similarly, the second protrusion snap 74 is positioned and attached to the second strap portion 30 such that the second strap portion 30 may have to stretch slightly prior to engagement between the second protrusion snap 74 and the second receiving snap 72. The strap portions 28, 30 stretch around the thumb 12 to retain the thumb protector 14 on the thumb 12.

This embodiment of the present invention also defines an opening 66b (see FIGS. 11 and 12) at the second distal ends 24 of the layers 16, 18. The opening 66b is sized and configurable to fit over large and small thumbs. The extent to which the opening 66b may receive different size thumbs 12 is limited by the elasticity of the layers 16, 18. The opening 66b defines a circumference and is preferably smaller compared to an outside diameter of the thumb 12. In this way, a retainment force is created which circumscribes the thumb 12. The retainment force may be created because the material is elastic and will stretch to fit (i.e., configurable) around the thumb 12. The frictional forces between the thumb skin and the layer material provide frictional forces so that the protector 14 is retained on the thumb 12. Additionally, as discussed above, the receiving and protrusion snaps 68, 72, 70, 74 are positioned on the layers 16, 18 such that when engaged and the protector 14 is placed on the thumb 12, the first and second strap portions 28, 30 will be positioned beyond the first joint 50 of the thumb 12. In this regard, the circumference of the opening 66b may have to be enlarged to a greater extent than the first joint 50 for the protector 14 to dislodge from the thumb 12 and provides an added force to retain the protector 14 on the thumb 12.

Additional modifications and improvements of the present invention may also be apparent to those of ordinary skill in the art. For example, it should be understood that the protector of the present invention may be fabricated in a variety of colors or otherwise made to bear ornamental designs, logos, web addresses, trademarks, phone numbers or other types of indicia to make the same more visually appealing. Along these lines, it is contemplated that the protector may be packaged and marketed as a collectors item or otherwise offered in connection with specific video games or video systems. Moreover, it is contemplated that the protector of the present invention may be fabricated in a variety of standardized sizes to thus enable the same to fit a specific type of digit, and in particular a variety of thumb sizes. Still further, the protector may be provided with one or more apertures to facilitate ventilation within the protector. Thus, the particular combination of parts and steps described and illustrated herein is intended to represent only certain embodiments of the present invention, and is not intended to serve as limitations of alternative devices and methods within the spirit and scope of the invention.

1. An appendage protector for protecting the thumb of a wearer of the protector, the protector comprising:
a. an attachment mechanism;
b. an upper layer defining first and second distal ends, the upper layer being attached to the attachment mechanism;
c. a lower layer defining first and second distal ends and a longitudinal axis, the lower layer being connectable to the upper layer at first distal ends of the lower and upper layers, the lower layer being connectable to the upper layer at second distal ends of the lower and upper layers with the attachment mechanism and forming an opening coaxially aligned with the longitudinal axis of the lower layer, said opening defining a cavity for receiving said thumb.

2. The protector of claim 1 wherein the upper and lower layers respectively define first and second edges that extend from the first distal ends to the second distal ends of the upper and lower layers, and the first and second edges of respective upper and lower layers form elongated apertures for aerating moisture excreted from the thumb.

3. The protector of claim 1 wherein the lower layer defines a contact portion on an external surface of the lower layer and has a tacky material adhered thereto.

4. The protector of claim 3 wherein the contact portion is cleanable such that the tacky material may be removed after use and additional tacky material may be applied thereto.

5. The protector of claim 1 wherein the layers are fabricated from stretchable material such that the lower layer may conform to a shape of the thumb.

6. The protector of claim 5 wherein the stretchable material is neoprene.

7. The protector of claim 6 wherein the neoprene material is approximately 1/8″ thick.

8. The protector of claim 1 wherein the attachment mechanism maintains the upper and lower layers in a closed position around the thumb and retains the protector about the thumb.

9. The protector of claim 8 wherein the attachment mechanism is connected to the upper and lower layers and has an engaged position such that second distal ends of the upper and lower layers define an opening for receiving the thumb, and a circumference of the opening being less than a diameter of the thumb.

10. The protector of claim 8 wherein the attachment mechanism is selected from the group consisting of hook and loop, button and button hole, buckle, and snaps.

11. The protector of claim 8 wherein the lower layer defines first and second strap portions that is extendable from an underside of the thumb to the upperside of the thumb, and the upper and lower layers are attachable at the second distal ends with the attachment mechanism.

12. The protector of claim 8 wherein the attachment mechanism is a hook and loop and the lower layer defines first and second strap portions, a first strip of loops being attached to an external surface of the upper layer at the second distal end, first and second strips of hooks being attached to external and internal layers, respectively, of the lower layer on the first strap portion such that the first strip of hooks is attachable to the first strip of loops, and a second strip of loops being attached to the internal layer of the second strap portion such that the second strip of loops is attachable to the second strip of hooks.

13. The protector of claim 8 wherein the attachment mechanism is snaps, and the lower layer defines first and second strap portions, a first receiving snap being attached to an external surface of the upper layer at the second distal end and a first protrusion snap being attached to the first strap portion on an internal surface and attachable to the first receiving snap, and a second receiving snap being attached to an external surface of the first strap portion and a second protrusion snap being attached to the internal surface of the second strap portion and attachable to the second receiving snap.
